(19) United States
(12) Patent Application Publication
(10) Pub. No.: US 2022/0313501 A1
(43) Pub. Date: Oct. 6, 2022

(54) LEAKPROOF PERIOD PANTIES FOR WEAR AT HOME AND DURING SLEEP

(71) Applicant: Kairui HE, Zhongshan (CN)

(72) Inventor: Kairui HE, Zhongshan (CN)

(21) Appl. No.: 17/243,603

(22) Filed: Apr. 29, 2021

(30) Foreign Application Priority Data
Apr. 6, 2021 (CN) 202110366536.5

(51) Int. Cl. A61F 13/496; A61F 13/494; A61F 13/49

(52) U.S. Cl. CPC A61F 13/4963; A61F 13/4946; A61F 13/49011; A61F 2013/530664

(57) ABSTRACT

Leakproof period panties for wear at home and during sleep include an outer layer. A leakproof structural layer is sewn on the inner side of the outer layer at the position of the crotch. The leakproof structural layer includes a lining layer. The top edge of the lining layer is sewn into the outer layer. A leakproof layer is sewn on the outer surface of the lining layer. An absorption layer is sewn on the bottom of the leakproof layer at the position of the crotch. The outer layer and the leakproof structural layer of the panties are combined to mimic a fake two-piece style. With the leakproof structural layer including a combination of a comfortable lining layer, an absorption layer for absorbing menstrual flow, and a leakproof layer for preventing seepage and lateral leakage, the period panties can serve as homewear or outerwear, while having an aesthetically attractive appearance.

### CROSS REFERENCE TO THE RELATED APPLICATIONS

This application is based upon and claims priority to Chinese Patent Application No. 202110366536.5, filed on Apr. 6, 2021, the entire contents of which are incorporated herein by reference.

### TECHNICAL FIELD

The present invention relates to the field of feminine hygiene products, and more particularly, to leakproof period panties for wear at home and during sleep.

### BACKGROUND

Menstruation is the regular, periodic discharge of blood from the uterus, typically occurring monthly. It is a physiological phenomenon unique to females. During a menstrual period, the associated physical changes take place, such as reduced excitability of the cerebral cortex and decreased resistance.

At present, menstruating females typically manage menstruation in two ways as follows. The first way is to conventionally wear underwear with sanitary napkins, in which case bed sheets may become stained due to menstrual flow caused by the change in sleeping posture throughout the night. The other way is to wear special menstrual underwear. Such menstrual underwear, however, is typically uncomfortable, lacks absorptive function, and can neither suitably serve as homewear nor avoid lateral leakage during sleep. It is, therefore, highly desirable to provide comfortable feminine period underwear suitable for wear at home and during sleep to avoid possible embarrassment and discomfort during menstrual periods.

### SUMMARY

### 1. Technical Problem to be Solved

In view of the problems identified in the prior art, an objective of the present invention is to provide leakproof period panties for wear at home and during sleep. According to the present invention, an outer layer and a leakproof structural layer of the panties are combined to mimic a two-piece style. With the leakproof structural layer including a combination of a comfortable lining layer, an absorption layer for absorbing menstrual flow, and a leakproof layer for preventing seepage and lateral leakage, the leakproof period panties for wear at home and during sleep can serve as homewear or outerwear for females during menstrual periods, while having an aesthetically attractive appearance.

### 2. Technical Solution

To solve the above-mentioned technical problems, the present invention adopts the following technical solution.

Leakproof period panties for wear at home and during sleep include an outer layer. A leakproof structural layer is sewn on the inner side of the outer layer at the position of the crotch. The leakproof structural layer includes a lining layer. The top edge of the lining layer is sewn into the outer layer. A leakproof layer is sewn on the outer surface of the lining layer. An absorption layer is sewn on the bottom of the leakproof layer at the position of the crotch. The outer layer serves as the outer main body, and the leakproof structural layer is configured to absorb menstrual flow. The lining layer is attached to the skin during wear. The absorption layer can absorb menstrual flow. The leakproof layer can further prevent seepage of the menstrual flow. Thus, females can wear the panties at home or during sleep to avoid embarrassment and discomfort during their menstrual periods.

Further, the top of the leakproof structural layer is sewn on the inner side of the outer layer along the waistline. The main body portion of the leakproof structural layer is suspended within the outer side of the panties. In this way, the outer layer and the leakproof structural layer of the panties are innovatively combined to mimic a fake two-piece style.

Further, a waist band is sewn on the top of the outer layer. The waist band is a fabric band internally provided with an elastic band or an elastic woven band. The waist band is relatively wide to avoid pressing against the skin of the waist during long-term wear. The fabric band internally provided with the elastic band or the elastic woven band provides improved comfort for females during wear.

Further, the lining layer, the absorption layer and the leakproof layer are sewn in a superimposed manner. The lining layer, the absorption layer and the leakproof layer are closely sewn together to form an independent integrated structure and thus can be distinguished from the outer layer that is commonly sewn from a single side.

Further, the lining layer is woven from a skin-friendly soft fabric. The absorption layer is mainly woven from a blend of super absorbent fiber materials. The leakproof layer is formed from a composite of thermoplastic polyurethane and a fabric. The lining layer woven from the skin-friendly soft fabric matches the skin and is thus more comfortable during wear. The absorption layer woven from a blend of the super absorbent fiber materials is highly effective in absorption, and is not prone to pilling even after long-term use. The leakproof layer has a moderate thickness and thus can maximally prevent seepage without affecting comfort.

Further, the bottom of the outer layer is hemmed by using a flat lock machine or a lockstitch sewing machine to achieve a good hemming effect without thread residue, which makes the panties more comfortable during wear.

### 3. Advantages

Compared with the prior art, the present invention has the following advantages.

(1) According to the present invention, the outer layer and the leakproof structural layer of the panties are combined to mimic a fake two-piece style. With the leakproof structural layer including a combination of a comfortable lining layer, an absorption layer for absorbing menstrual flow, and a leakproof layer for preventing seepage and lateral leakage, the leakproof period panties for wear at home and during sleep can serve as homewear or outerwear for females during menstrual periods, while having an aesthetically attractive appearance.

(2) The panties are comfortable and function to prevent leakage during menstrual periods. The panties can be worn alone for a lone time during sleep to replace the need for sanitary napkins without being repeatedly changed, while greatly reducing the chance of staining the bed sheet. Tailored with superb craftsmanship, style and fabric, the panties provided in the present invention are high in quality, washable, reusable, and more comfortable and cost-efficient compared with traditional menstrual underwear or sanitary napkins.

### BRIEF DESCRIPTION OF THE DRAWINGS

FIG. 1 is a structural schematic view of the present invention.

FIG. 2 is a cross-sectional view of the present invention.

FIG. 3 is a structural schematic view of a leakproof structural layer of the present invention.

### REFERENCE NUMERALS

1, outer layer; 2, leakproof structural layer; 201, lining layer; 202, leakproof layer; 203, absorption layer; and 3, waist band.

### DETAILED DESCRIPTION OF THE EMBODIMENTS

The technical solutions in embodiments of the present invention will be described below clearly and completely with reference to the drawings in the embodiments of the present invention. Apparently, the described embodiments are merely a part rather than all of the embodiments of the present invention. All other embodiments derived from the embodiments in the present invention by a person of ordinary skill in the art without creative efforts shall fall within the scope of protection of the present invention.

In the description of the present invention, it should be noted that orientations or position relationships indicated by terms “top”, “bottom”, “inner/inside”, “outer/outside”, and the like are orientations or position relationships as shown in the drawings, and these terms are just used to facilitate description of the present invention and simplify the description, rather than to indicate or imply that the mentioned apparatus or elements must have a specific orientation and must be established and operated in a specific orientation, and thus cannot be construed as a limitation to the present invention. Moreover, terms such as “first” and “second” are merely intended for the purpose of description, rather than to indicate or imply relative importance.

In the description of the present invention, it should be noted that, unless otherwise clearly specified and limited, the meanings of terms “installed/mounted”, “provided with”, “sleeved on/to” and “connection” should be understood in a board sense. For example, the “connection” may be a fixed connection, a removable connection, or an integral connection; may be a mechanical connection or an electrical connection; may be a direct connection or an indirect connection through an intermediate medium; or may be an internal communication between two components. A person of ordinary skill in the art may understand specific meanings of the foregoing terms in the present invention based on a specific situation.

### Embodiments

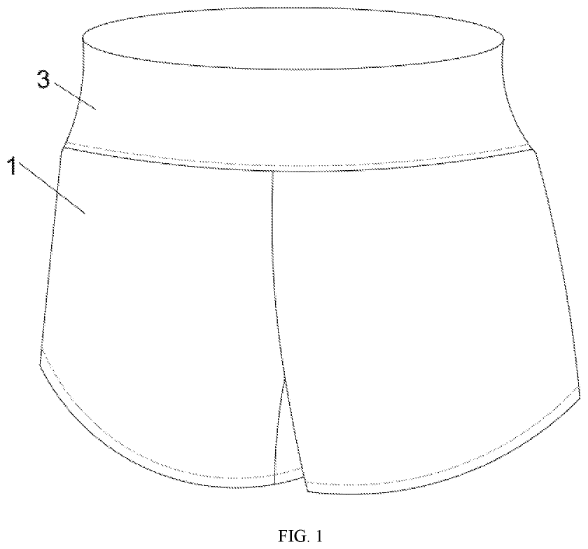

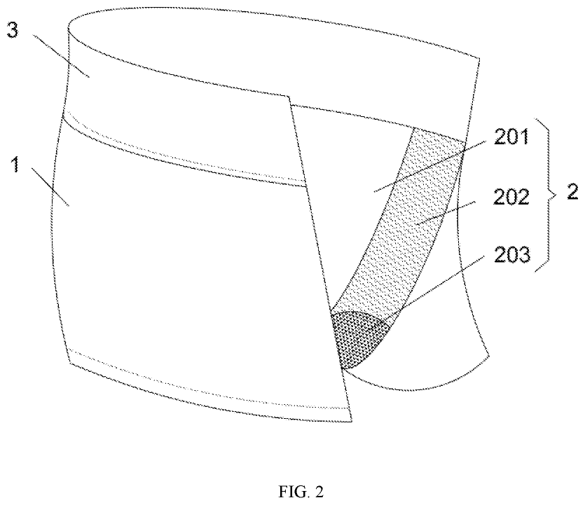

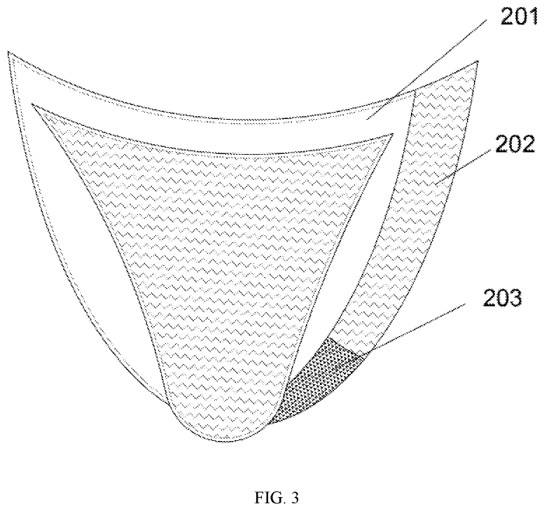

Referring to FIG. 1 to FIG. 3, leakproof period panties for wear at home and during sleep include the outer layer 1. The leakproof structural layer 2 is sewn on the inner side of the outer layer 1 at the position of the crotch. The leakproof structural layer 2 includes the lining layer 201. The top edge of the lining layer 201 is sewn into the outer layer 1. The leakproof layer 202 is sewn on the outer surface of the lining layer 201. The absorption layer 203 is sewn on the bottom of the leakproof layer 202 at the position of the crotch. The outer layer 1 serves as the outer main body, and the leakproof structural layer 2 is configured to absorb menstrual flow. The lining layer is attached to the skin during wear. The absorption layer 203 can absorb menstrual flow. The leakproof layer 202 can further prevent seepage of the menstrual flow. Thus, females can wear the panties at home or during sleep to avoid embarrassment and discomfort during their menstrual periods.

Referring to FIG. 1 to FIG. 3, the top of the leakproof structural layer 2 is sewn on the inner side of the outer layer 1 along the waistline. The main body portion of the leakproof structural layer 2 is suspended within the outer side of the panties. In this way, the outer layer 1 and the leakproof structural layer 2 of the panties are innovatively combined to mimic a fake two-piece style.

Referring to FIG. 1 to FIG. 3, the waist band 3 is sewn on the top of the outer layer 1. The waist band 3 is a fabric band internally provided with an elastic band or an elastic woven band. The waist band 3 is relatively wide to avoid pressing against the skin of the waist during long-term wear. The fabric band internally provided with the elastic band or the elastic woven band makes the waist band 3 more comfortable during wear.

Referring to FIG. 1 to FIG. 3, the lining layer 201, the absorption layer 203 and the leakproof layer 202 are sewn in a superimposed manner. The lining layer 201, the absorption layer 203 and the leakproof layer 202 are closely sewn together to form an independent integrated structure and thus can be distinguished from the outer layer 1 that is commonly sewn from a single side.

Referring to FIG. 1 to FIG. 3, the lining layer 201 is woven from a skin-friendly soft fabric. The absorption layer 203 is mainly woven from a blend of super absorbent fiber materials. The leakproof layer 202 is formed from a composite of thermoplastic polyurethane and a fabric. The lining layer 201 woven from the skin-friendly soft fabric matches the skin and is thus more comfortable during wear. The absorption layer 203 woven from a blend of the super absorbent fiber materials is highly effective in absorption, and is not prone to pilling even after long-term use. The leakproof layer 202 has a moderate thickness and thus can maximally prevent seepage without affecting comfort.

Referring to FIG. 1 to FIG. 3, the bottom of the outer layer 1 is hemmed by using a flat lock machine or a lockstitch sewing machine to achieve a good hemming effect without thread residue, which makes the panties more comfortable during wear.

With the outer layer 1 as the outer main body, the panties of the present invention are provided with the leakproof structural layer 2 to absorb menstrual flow. The lining layer is attached to the skin during wear. The absorption layer 203 can absorb menstrual flow. The leakproof layer 202 can further prevent seepage of the menstrual flow. The main body portion of the leakproof structural layer 2 is suspended such that the outer layer 1 and the leakproof structural layer 2 of the panties are innovatively combined to mimic a fake two-piece style. The waist band 3 is relatively wide to provide improved comfort for users during wear. The lining layer 201 woven from the skin-friendly soft fabric matches the skin and is thus more comfortable during wear. The absorption layer 203 woven from a blend of the super absorbent fiber materials is highly effective in absorption, and is not prone to pilling even after long-term use. The leakproof layer 202 has a moderate thickness and thus can maximally prevent seepage to avoid females' embarrassment and discomfort during their menstrual periods. The panties are convenient for wear at home, and are breathable and aesthetically attractive, while functioning to prevent leakage during menstrual periods. The panties can be worn alone for a long time during sleep to replace the need for sanitary napkins without being repeatedly changed, while reducing the chance of staining the bed sheet. The period panties suitable for wear at home and during sleep are leakproof, washable and reusable, and therefore are not only convenient but also practicable for females.

The foregoing are merely the preferred specific embodiments of the present invention, but the scope of protection of the present invention is not limited thereto. Any equivalent replacements or modifications made within the technical scope of the present invention by a person skilled in the art according to the technical solutions of the present invention and inventive concepts thereof shall fall within the scope of protection of the present invention.

What is claimed is:

1. Leakproof period panties for wear at home and during sleep, comprising an outer layer;
wherein
a leakproof structural layer is sewn on an inner side of the outer layer at a position of a crotch;
the leakproof structural layer comprises a lining layer;
a top edge of the lining layer is sewn into the outer layer;
a leakproof layer is sewn on an outer surface of the lining layer; and
an absorption layer is sewn on a bottom of the leakproof layer at the position of the crotch.

2. The leakproof period panties according to claim 1, wherein
a top of the leakproof structural layer is sewn on the inner side of the outer layer along a waistline; and
a main body portion of the leakproof structural layer is suspended within an outer side of the leakproof period panties.

3. The leakproof period panties according to claim 1, wherein
a waist band is sewn on a top of the outer layer, and
the waist band is a fabric band internally provided with an elastic band or the waist band is an elastic woven band.

4. The leakproof period panties according to claim 1, wherein
the lining layer, the absorption layer and the leakproof layer are sewn in a superimposed manner.

5. The leakproof period panties according to claim 1, wherein
the lining layer is woven from a skin-friendly soft fabric;
the absorption layer is woven from a blend of super absorbent fiber materials; and
the leakproof layer is formed from a composite of thermoplastic polyurethane and a fabric.

6. The leakproof period panties according to claim 1, wherein
a bottom of the outer layer is hemmed by using a flat lock machine or a lockstitch sewing machine.
